(19) United States
(12) Patent Application Publication
(10) Pub. No.: US 2020/0015727 A1
(43) Pub. Date: Jan. 16, 2020

(54) VIRTUAL FORCED FIXATION

(71) Applicant: Envision Solutions LLC, Bonita Springs, FL (US)

(72) Inventor: Andrew Maskery, Bonita Springs, FL (US)

(21) Appl. No.: 16/512,810

(22) Filed: Jul. 16, 2019

Related U.S. Application Data
(60) Provisional application No. 62/698,571, filed on Jul. 16, 2018.

(51) Int. Cl. A61B 5/16; A61H 5/00; G06F 3/01; A61B 3/113

(52) U.S. Cl. CPC A61B 5/163; A61H 5/00; G06F 3/013; A61H 2205/024; A61H 2201/1207; A61H 2201/5043; A61B 3/113

(57) ABSTRACT

A computer-implemented method, system, and computer program product where a processor(s) displays, at a first location in an interface of a computing device, image(s) comprising a fixation point. The processor(s) obtains, from an eye tracking device, a first indication that a user whose eye movement is being tracked has at least one eye focused on the fixation point. The processor(s) renders, at a second location in the interface, a visual stimulus to stimulate a predefined location in a retina of the at least one eye. The processor(s) obtains, from the eye tracking device, a second indication that a user does not have the at least one eye focused on the fixation point. The processor(s) identifies a third location, a focal point of the user. The processor(s) reorients the image(s) such that the fixation point is at the third location and the stimulus is moved relative to the fixation point.

### CROSS-REFERENCE TO RELATED APPLICATIONS

This application claims priority to U.S. Provisional Application No. 62/698,571 filed Jul. 16, 2018, entitled, “VIRTUAL FORCED FIXATION” which is incorporated herein by reference in its entirety.

### FIELD OF INVENTION

The invention relates generally to systems and methods that enable vison training through software and/or hardware fixation controls.

### BACKGROUND OF INVENTION

Improved vision training is ideally enables by the repeated visual stimulation of a specific area of a retina within an eye. An impediment to this training is the movement of the eye. When an individual participating in a given visual training exercises either voluntarily and/or involuntarily moves his or her eye (e.g., changes his or her point of focus) this repeated stimulation is complicated and generally, not possible and thus, the efficacy of the training is compromised.

### SUMMARY OF INVENTION

Shortcomings of the prior art are also overcome and additional advantages are provided through the provision of a method to stimulate a consistent part of a retina of a user, the method comprising displaying, by one or more processors, at a first location in an interface of a computing device, one or more images comprising a fixation point; obtaining, by the one or more processors, from an eye tracking device communicatively coupled to the one or more processors, a first indication that a user whose eye movement is being tracked by the eye tracking device has at least one eye focused on the fixation point; based on the first indication, rendering, by the one or more processors, at a second location in the interface, a visual stimulus, wherein the second location is a fixed vertical distance and a fixed horizontal distance from the first location, and wherein based on being rendered at the second location, the stimulus stimulates a predefined location in a retina of the at least one eye of the user when the user has at least one eye focused on the fixation point; obtaining, by the one or more processors, from the eye tracking device, a second indication that a user whose eye movement is being tracked by the eye tracking device does not have the at least one eye focused on the fixation point; identifying, by the one or more processors, based on utilizing the eye tracking device, a third location in the interface, wherein the third location comprises a focal point of the at least one eye of the user, in the interface; and based on identifying the third location, reorienting, by the one or more processors, the one or more images such that the fixation point is at the third location and the stimulus is moved relative to the fixation point, maintaining the fixed vertical distance and the fixed horizontal distance.

Systems and methods relating to one or more aspects of the technique are also described and may be claimed herein. Further, services relating to one or more aspects of the technique are also described and may be claimed herein.

Additional features are realized through the techniques of the present invention. Other embodiments and aspects of the invention are described in detail herein and are considered a part of the claimed invention.

### BRIEF DESCRIPTION OF DRAWINGS

One or more aspects of the present invention are particularly pointed out and distinctly claimed as examples in the claims at the conclusion of the specification. The foregoing and objects, features, and advantages of one or more aspects of the invention are apparent from the following detailed description taken in conjunction with the accompanying drawing.

FIG. 1 is an example of a technical architecture comprising aspects of some embodiments of the present invention.

FIG. 2 a workflow illustrating certain aspects of an embodiment of the present invention.

FIGS. 3A-3B illustrate certain aspects of some embodiments of the present invention.

FIG. 4 is a workflow illustrating certain aspects of an embodiment of the present invention.

FIGS. 5A-5C illustrate certain aspects of some embodiments of the present invention.

FIG. 6 is a workflow illustrating certain aspects of an embodiment of the present invention.

FIG. 7 depicts a computer system configured to perform an aspect of an embodiment of the present invention.

FIG. 8 depicts a computer program product incorporating one or more aspects of the present invention.

### DETAILED DESCRIPTION OF THE INVENTION

Aspects of the present invention and certain features, advantages, and details thereof, are explained more fully below with reference to the non-limiting examples illustrated in the accompanying drawings. Descriptions of well-known materials, fabrication tools, processing techniques, etc., are omitted so as not to unnecessarily obscure the invention in detail. It should be understood, however, that the detailed description and the specific examples, while indicating aspects of the invention, are given by way of illustration only, and not by way of limitation. Various substitutions, modifications, additions, and/or arrangements, within the spirit and/or scope of the underlying inventive concepts will be apparent to those skilled in the art from this disclosure. The terms software and program code are used interchangeably throughout this application.

As understood by one of skill in the art, to enable vision training, one would ideally want to stimulate a specific area of the retina within the eye. If the eye moves, one cannot stimulate the same part of the retina. In order to overcome this and allow vision training when eye movement occurs, program code executing on one or more processors, in embodiments of the present invention, renders a simulation (e.g., an image on a display) and when a viewer moves his or her eye(s), the program code moves the stimulation by the same amount that the eye(s) moved, so that the stimulation is back training the same area of the retina it was before the eye movement occurred.

Embodiments of the present invention include program code executing on one or more processors that enable eye tracking technologies, that when combined with a visual display (graphical user interface on a computing device) and/or an interface of a virtual reality (VR) device, including but not limited to a headset, enable vision training with fixation control. In embodiments of the present invention, program code executing on at least one processor moves an object within the view of a user through a computing interface, including but not limited to a graphical user interface on a screen and/or within a viewer of a VR device. As understood by one of skill in the art, VR devices include, but are not limited to, virtual reality headsets, which are head-mounted devices that provides a virtual reality experience for the wearer. A VR headset can comprise a stereoscopic head-mounted display, which provides separate images for each eye and head motion tracking sensors (e.g., gyroscopes, accelerometers, magnetometers, structured light systems, etc.). VR headsets utilized in embodiments of the present invention can also include integrated eye tracking sensors. Other computing interfaces utilized in embodiments of the present invention include various user interfaces, including screens of various computing devices, which render graphical user interfaces to the user. In embodiments of the present invention, when the program code moves the object within the view of a user (e.g., the view on an interface including a screen and/or a head-mounted display), the program code forces the user to fixate on a given portion of the image. The program code manipulates the placement of an image or a portion of an image in the interface, based on obtain the focus point of a user's eyes and displaying the image at the user's focus point, even if the user moves his or her eyes and attempts to focus on a different part of the image. Thus, the program code moves the image to maintain the same portion of the image at a location in the interface that coordinates with the focus point of the user. By shifting an image on an interface to comport with a focus point of a user, program code in embodiments of the present invention can provide various training exercises and vison assessments of the user, via the interface. Because the program code identifies and isolates are area of an eye of the user, the training and vison assessments that the program code displays to the user, through the interface, are more effective.

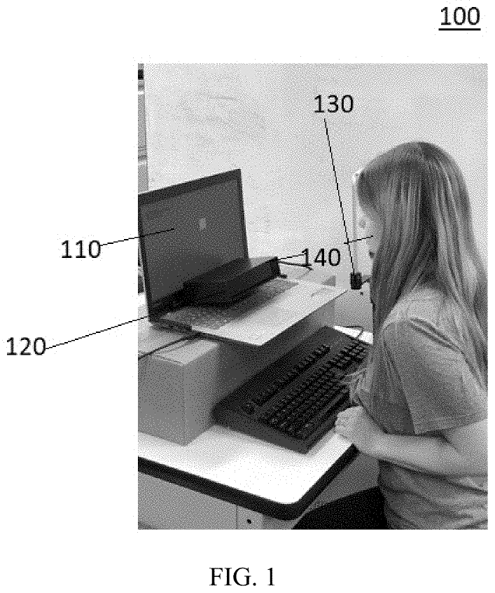

Embodiments of the present invention are inextricably tied to computing and are directed to a practical application. FIG. 1 illustrates these aspects. As illustrated in FIG. 1, embodiments of the present invention utilize a computing infrastructure 100 to train and assess the vison of a user. As illustrated in FIG. 1, an infrastructure 100 utilized to perform aspects of the present invention include a user interface 110 (e.g., a display, including but not limited to a head-mounted display), an eye movement tracking device 120, which can be integrated into a computing device comprising the interface 110, including a headset, and program code executing on one or more processors to display one or more images in the interface 110 and utilize the eye movement tracking device 120 to locate a focus or focal point of the user on the displayed one or more images. Eye movement and focus tracking within the context of embodiments of the present invention, as performed by the eye movement tracking device 120, refers to a process of measuring either the point of gaze (where one is looking) or the motion of an eye relative to the head. As explained herein, the program code in embodiments of the present invention interacts with and/or includes eye tracking of a point of gaze (also referred to herein as a focus point) of a user. The eye movement tracking device 120 is utilized by the program code to measure eye positions and eye movement. Method utilizes within the eye movement tracking device 120 to measure eye movement include, but are not limited to, capturing video images of the eyes of a user (e.g., the eye movement tracking device 120 can include a video capture device) and extracting eye position from the images, utilizing search coils, and/or generating an electrooculogram (by utilizing electrooculography, a technique for measuring the corneo-retinal standing potential that exists between the front and the back of the human eye). Depending upon factors including the placement of the interface 110 (e.g., whether the display is head-mounted or at a physical distance 140 from the user), the eye movement tracking device 120 can be an eye-attached tracking device (e.g., a special contact lens with an embedded mirror or magnetic field sensor,), an optical tracking device in a head-mounted display (e.g., an eye-tracking head-mounted display where a light emitting diode (LED) light source (gold-color metal) on the side of the display lens, and a camera under the display lens), an optical device that utilizes video recording, and/or an electrical potential measurement optical device (e.g., electric potentials measured with electrodes placed around the eyes). In embodiments of the present invention where the eye movement tracking device 120 is part of a head-mounted display (e.g., the interface 110), the embodiments can include a stationary 130 object to position the face of the user, such that the position in three dimensional space of the user's eyes, relative to the display 110, remain constant. An example of such a stationary object 130, which is illustrated in FIG. 1, is a chin rest and face framing device. The chin rest and the face framing device together keep the face/head of a user at a constant position within three dimensional (physical) space. In some embodiments of the present invention, when the interface 110 is a head-mounted display, the program code can track the position of the user's head (and eyes) in three dimensional space by utilizing the motion tracking sensors (e.g., gyroscopes, accelerometers, magnetometers, structured light systems, etc.) integrated into the head-mounted display, rather than physically constricting movement via a physical stationary object 130. Thus, when utilizing a head mounted display, although the orientation of the user can change in three dimensional space, the program code can utilize the motion tracking sensors within the head-mounted display to virtually maintain the position of the user's head relative to the interface 110. Thus, in embodiments of the present invention, both hardware and software elements are utilized to effectively train the vision of a user. Thus, not only are aspects of embodiments of the present invention inextricably tied to computing, the program tracking and manipulating images in an interface to maintain focus of a user, to effectively train the eyesight of the user, represents a practical application.

Embodiments of the present invention provide significant advantages over existing approaches to eye training. In general, to enable vision training, one would ideally want to stimulate a specific area of the retina within the eye. If the eye moves, one cannot stimulate the same part of the retina. In order to overcome this and allow vision training when eye movement occurs, in embodiments of the present invention, the program code moves the stimulation (the image and/or images rendered by the program code in the interface 110, FIG. 1) by the same amount that the eye moves, so that the stimulation is back training the same area of the retina it was before the eye movement occurred. Thus, rather than rely on an individual to maintain the positioning of his or her eyes, as well as the focus, embodiments of the present invention represent a significant improvement by forcing this constant positioning.

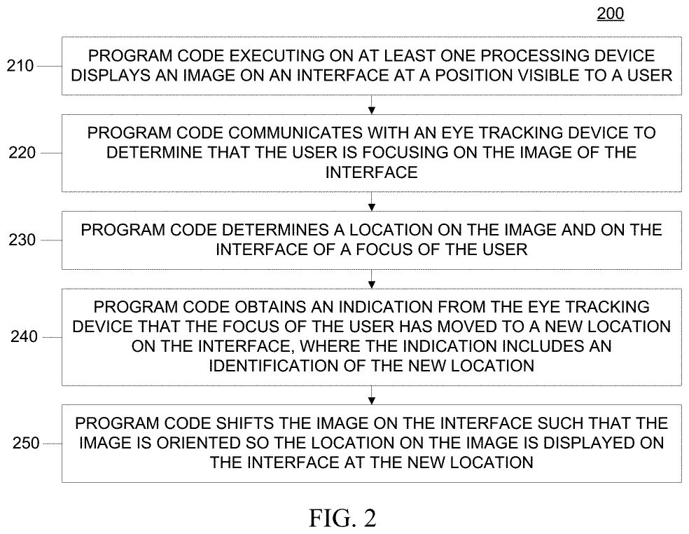

FIG. 2 is a workflow 200 that describes various aspects of some embodiments of the present invention. In embodiments of the present invention, program code executing on at least one processing device displays an image on an interface at a position visible to a user (210). The program code communicates with an eye tracking device communicatively coupled to the at least one processing device to determine that the user is focusing on the image of the interface (220). In some embodiments of the present invention, the eye tracking device comprises sensors that are communicatively coupled to the at least one processor executing the program code. In some embodiments of the present invention, the eye tracking device captures images and/or video of the eyes of the user and the program code analyzes these images and/or video to determine if and when the user has focused on the image. The program code determines a location on the image and in the interface of a focus of the user (230). The program code obtains an indication from the eye tracking device that the focus of the user has moved to a new location in the interface, where the indication includes an identification of the new location (240). For example, in some embodiments of the present invention, the identification of the new location could include coordinates on the display for where the user is now focusing. Based on obtaining the indication, the program code shifts the image in the interface such that the image is oriented so the location on the image is displayed in the interface at the new location (250). Thus, as seen from this workflow 200, aspects of some embodiments of the present invention include program code that creates a situation where a user would not be able to look away from the location on the image, as the program code obtains the focus point of a user's eyes and displays the location on the image, in the interface, at the user's focus point, even if the user moves the user's eyes and attempts to focus on a different part of the image.

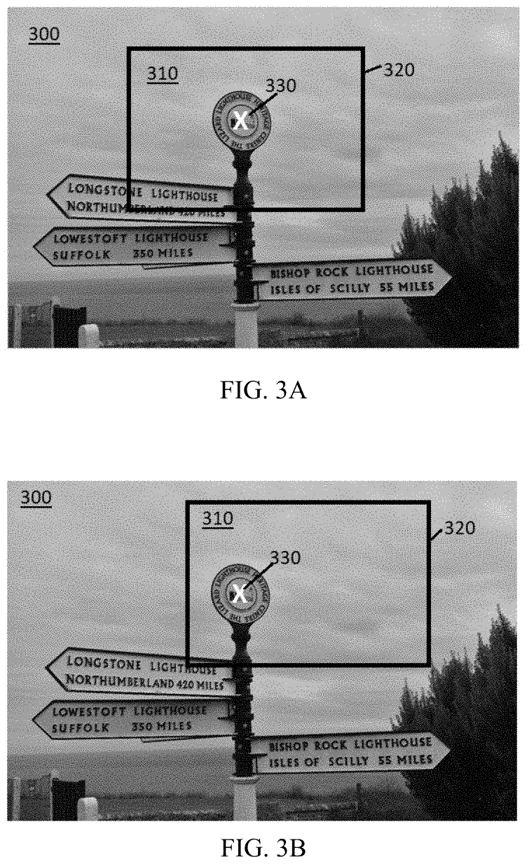

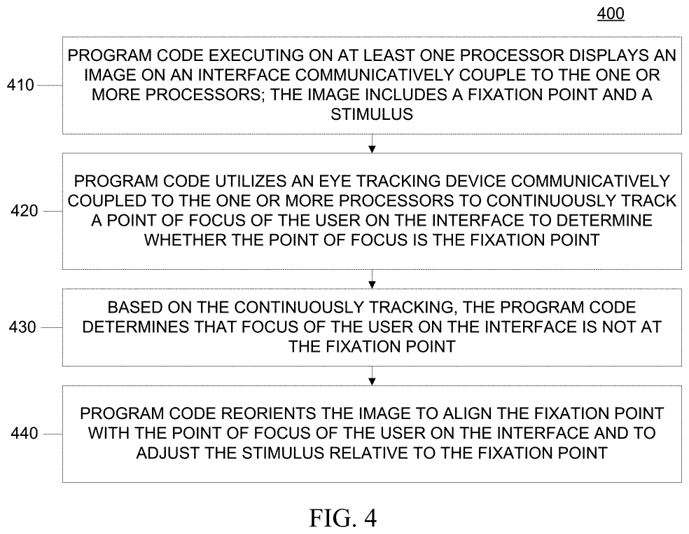

FIGS. 3A-3B illustrate the image shifting aspect (e.g., FIG. 2, 250) of FIG. 2. FIGS. 3A-3B displays a portion 310 of a given image 300 on a display. FIGS. 3-4, for illustrative purposes only, show the entirety of a given image 300 and the portion 310, which is what is displayed to a user in an interface, at two different times. FIG. 3A shows the portion of the image 310 displayed at a first time, and FIG. 3B shows the portion of the image 310 displayed at a second time, where the second time, in this non-limiting example, is after the first time. The rectangle 320 delineates the portion 310 displayed at each time, in the interface (e.g., FIG. 1, 110) from the remainder of the image 300. The “X” designation in FIGS. 3A-3B is not displayed to a user and is an indication in FIGS. 3A-3B, for illustrative purposes only, of a location of the focus 330 of the user, when the user looks at the display (interface). As discussed in reference to FIG. 2, the program code determines the focus of the user by utilizing an eye tracking device. FIG. 3A depicts the focus 330 at a first time and FIG. 3B depicts the focus at a second time. The second time is after program code obtains an indication from the eye tracking device that the focus of the user has moved to a new location in the interface, where the indication includes an identification of the new location (FIG. 2, 240). In some embodiments of the present invention, the program code displays the “X” or another indication of the focus location, to the user, via a user interface. In this non-limiting example, in FIG. 3B, the program code has determined that the eye(s) of the user have moved to look at the lower right part of the interface (e.g., FIG. 1, 110). Because this movement was detected by the eye tracking equipment in an embodiment of the present invention and provided to the program code, the program code moves portion 310 of the image being displayed to the user, so that the program code displays, to the user, the same part of the image, at the newly determined focus or fixation point. Hence, as the fixation of the eye changes, the program code changes the orientation in the interface of the portion of the image visible to the user.

As illustrated in FIGS. 3A-3B, the program code had shifted the image at the second time such that the same region of the portion 310 of the image displayed in the interface is at the focus 330 point of the user in the interface. Thus, the fixation point of the screen (interface) displays a consistent region of the portion 310 of the image to the user. In this example, the present invention includes program code that creates a situation where a user would not be able to look away from the round sign, as the program code would obtain the focus point of a user's eyes and display the image at the user's focus point, even if the user moves his or her eyes and/or attempts to focus on a different part of the displayed portion of the image 310. The program code, by utilizing the eye tracking technology in embodiments of the present invention, can move a region of an image containing stimulus, so that even with eye movement, the stimulus is affecting the same area of the retina of the user.

FIG. 4 is a workflow 400 that illustrates various aspects of some embodiments of the present invention. In some embodiments of the present invention, program code executing on at least one processor displays an image on an interface communicatively couple to the one or more processors; the image includes a fixation point and a stimulus (410). The image can comprise an eye training exercise. The program code positions the fixation point to enable the user to focus on a particular location in the interface. When the user is focused on this fixation point, the stimulus is aligned with a portion of the retina of the user such that the stimulus can stimulate this portion of the retina. In some embodiments of the present invention, the stimulus is an image which can be dynamic. In an embodiments of the present invention, the program code utilizes an eye tracking device communicatively coupled to the one or more processors to continuously track a point of focus of the user in the interface to determine whether the point of focus is the fixation point (420). Based on the continuously tracking, the program code determines that focus of the user in the interface is not at the fixation point (430). The program code reorients the image to align the fixation point with the point of focus of the user in the interface and to adjust the stimulus relative to the fixation point (440). In some embodiments of the present invention, the fixation point is not visible to the user. In some embodiments of the present invention, the stimulus is not populated in the interface by the program code until the program code determines, by utilizing the eye tracking device, that the user is focused on the fixation point. In some embodiments of the present invention, the fixation point is a visual fixation cue in the image to enable the user to attempt to focus, initially, on the image. In embodiments of the present invention, in reorienting the image, the program code maintains a relative position of the fixation point and the stimulus to each other.

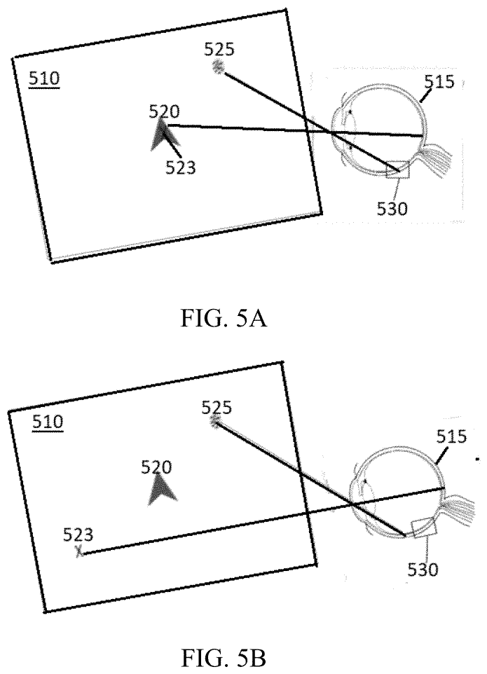

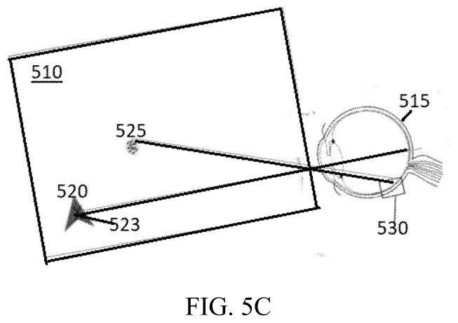

FIGS. 5A-5C demonstrate how the program code moving an image on a display (e.g., FIG. 2, 250) isolates the stimulation of the eye of the user/viewer to a given region, thus allowing the content of the display (e.g., images comprising a training exercise), to effectively train the eye of the user/viewer. The rectangle in FIGS. 5A-5C is an interface 510 (e.g., head-mounted display, monitor of a computing device, etc.) that the user is using an eye 515 to look at. A fixation cue 520 (represented by an arrow) is where the user should focus his or her gaze in order to benefit from a vision training exercise being provided by the program code in the interface 510. In order to train the vision of the user, the program code displays stimulus 525 in the interface 510. The stimulus 525 should be positioned in the interface 510 to align with a predetermined area of the retina 530 of the user, such that the predetermined area of the retina 530 is stimulated when the eye 515 is focused on the fixation cue 520. FIG. 5A depicts an alignment of the elements displayed such that the eye 515 is stimulated by the vision training exercise in the interface 510.

Referring to FIG. 5A, the user's eye 515 is fixating/focusing on a location 523 in the interface 520 that is the fixation cue 520. The predetermined area of the retina 530 is aligned with the stimulus 525, based on the focus of the eye 515. The program code can monitor this alignment by communicating with an eye tracking device, including obtaining videos from a video capture device and determining the alignment of the focus of the eye 515 based on the media received.

Turning to FIG. 5B, in some embodiments of the present invention, the program code determines, based on obtaining data from an eye tracking device, which can be external to and/or embedded in the interface 510, that the user is no longer looking at the fixation cue 520. The program code determined that the location 523 is not the fixation cue 230, as illustrated in FIG. 5B. When the user is not looking at the fixation cue 520, the training exercise provided by the program code cannot stimulate the predetermined area of the retina 530 and would impact a different area of the eye 515 of the user. In some embodiments of the present invention, the program code monitors data from the eye tracking device continuously. In other embodiments of the present invention, the program code obtains this data intermittently at varied and/or fixed intervals. In some embodiments of the present invention, the timing of the communications between the program code and the eye tracking device are configurable by a user.

Once the program code determines that the user is no longer looking at the fixation cue 520, the program code immediately (i.e., at a speed that is transparent to the user), can change the orientation of the training exercise being provided by the program code in the interface 510. Thus, the amount of time that the orientation of the interface 510 relative to the eye 515, as depicted in FIG. 5B, persists in minimal. The program code determines that the focus has changed and corrects the orientation of the training exercise to maintain continuity of training. This continuity enables the effective (seamless) training of the eyes of the user by maintaining the stimulus.

Referring to FIG. 5C, based on determining the eye has moved, and therefore the area of the retina to be stimulated is no longer aligned with the program code reorients the training exercise being provided by the program code in the interface 510, with the assistance of the eye (movement) tracking device, such that the user's eye 515 is fixating on a location 523 comprising the fixation cue 520. Because the training exercise (e.g., image(s)) is provided in the interface 510 in an orientation where the user's eye 515 is fixating on the fixation cue 520, the program code can position the stimulus 525 in the interface 510 to align with a predetermined area of the retina 530. Thus, as depicted in the non-limiting example of FIG. 5C, the program code has moved the image in such a way that the fixation cue 520 is fixated upon by the user's eye 515 and the stimulus in the interface 510 is aligned with the predetermined area of the retina 530, thus maintaining, in FIG. 5C, the same relative positioning as in FIG. 5A.

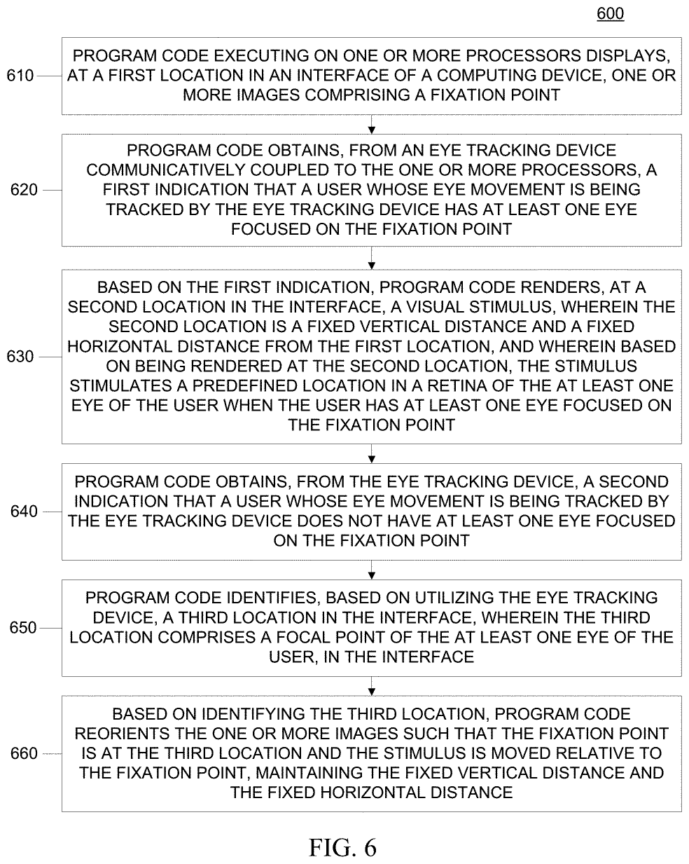

FIG. 6 is a workflow 600 that depicts certain aspects of some embodiments of the present invention. In some embodiments of the present invention, program code executing on one or more processors displays, at a first location in an interface of a computing device, one or more images comprising a fixation point (610). The program code obtains, from an eye tracking device communicatively coupled to the one or more processors, a first indication that a user whose eye movement is being tracked by the eye tracking device has at least one eye focused on the fixation point (620). Based on the first indication, the program code renders, at a second location in the interface, a visual stimulus, wherein the second location is a fixed vertical distance and a fixed horizontal distance from the first location, and wherein based on being rendered at the second location, the stimulus stimulates a predefined location in a retina of the at least one eye of the user when the user has at least one eye focused on the fixation point (630). The stimulus can be a vison training sequence, including but not limited to the rendering of dots at the second location, for a given period of time, including but not limited to, 500 milliseconds or 500 microseconds. The dots (or other images) can pulse (appear and disappear) for this span of time, as the eye training.

Returning to FIG. 6, in some embodiments of the present invention, the program code obtains, from the eye tracking device, a second indication that a user whose eye movement is being tracked by the eye tracking device does not have at least one eye focused on the fixation point (640). The program code identifies, based on utilizing the eye tracking device, a third location in the interface, wherein the third location comprises a focal point of the at least one eye of the user, in the interface (650). Based on identifying the third location, the program code reorients the one or more images such that the fixation point is at the third location and the stimulus is moved relative to the fixation point, maintaining the fixed vertical distance and the fixed horizontal distance (660).

Embodiments of the present invention include a computer-implemented method, a computer program product, and a computer system, where program code executed by one or more processors, displays, at a first location in an interface of a computing device, one or more images comprising a fixation point. The program code obtains, from an eye tracking device communicatively coupled to the one or more processors, a first indication that a user whose eye movement is being tracked by the eye tracking device has at least one eye focused on the fixation point. Based on the first indication, rendering, at a second location in the interface, a visual stimulus, where the second location is a fixed vertical distance and a fixed horizontal distance from the first location, and where based on being rendered at the second location, the stimulus stimulates a predefined location in a retina of the at least one eye of the user when the user has at least one eye focused on the fixation point. The program code obtains, from the eye tracking device, a second indication that a user whose eye movement is being tracked by the eye tracking device does not have the at least one eye focused on the fixation point. The program code identifies, based on utilizing the eye tracking device, a third location in the interface, wherein the third location comprises a focal point of the at least one eye of the user, in the interface. Based on identifying the third location, the program code reorients the one or more images such that the fixation point is at the third location and the stimulus is moved relative to the fixation point, maintaining the fixed vertical distance and the fixed horizontal distance.

In some embodiments of the present invention, the interface comprises a head-mounted display.

In some embodiments of the present invention, obtaining the first indication and obtaining the second indication is based on the one or more processors continuously monitoring the eye tracking device.

In some embodiments of the present invention, obtaining the first indication and obtaining the second indication is based on the one or more processors intermittently obtaining data from the eye tracking device.

In some embodiments of the present invention, the program code obtaining the first indication comprises: the program code obtaining video data captured by the eye tracking device; the program code analyzing the video data to determine a focal point of the at least one eye in the interface; the program code comparing the focal point to the fixation point; and the program code determining that the focal point is equivalent to the fixation point

In some embodiments of the present invention, the program code rendering the visual stimulus comprises displaying one or more images in a manner selected from the group consisting of: displaying an the one or more images sequentially in the interface at the second location, and pulsing the one or more image on and off at the second location.

In some embodiments of the present invention, the stimulus is moved relative to the fixation point to a fourth location in the interface.

In some embodiments of the present invention, the program code, based on obtaining the second indication, stops the displaying of the one or more images.

In some embodiments of the present invention, based on reorienting the one or more images, the program code resumes the displaying of the one or more images at the fourth location.

In some embodiments of the present invention, the reorienting by the program code is timed such that the user cannot perceive the reorienting.

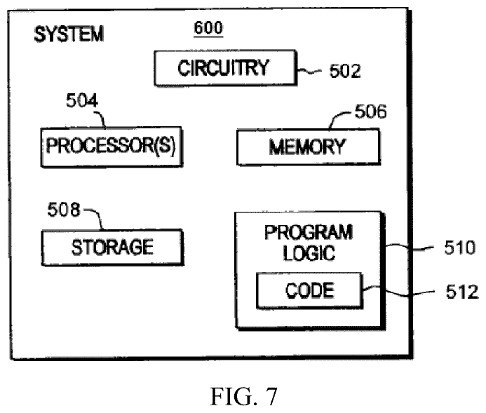

FIG. 7 illustrates a block diagram of a resource 600 in computer system, which can include one or more processors to execute the program code referred to throughout this disclosure. The computer system 600 can also include an eye (movement) tracking device. Returning to FIG. 7, the resource 600 may include a circuitry 502 that may in certain embodiments include a microprocessor 504. The computer system 600 may also include a memory 506 (e.g., a volatile memory device), and storage 508. The storage 508 may include a non-volatile memory device (e.g., EEPROM, ROM, PROM, RAM, DRAM, SRAM, flash, firmware, programmable logic, etc.), magnetic disk drive, optical disk drive, tape drive, etc. The storage 508 may comprise an internal storage device, an attached storage device and/or a network accessible storage device. The system 600 may include a program logic 510 including code 512 that may be loaded into the memory 506 and executed by the microprocessor 504 or circuitry 502.

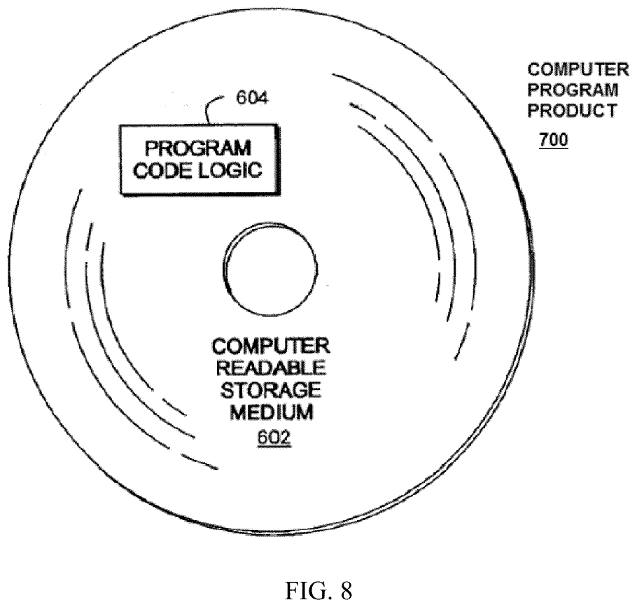

In certain embodiments, the program logic 510 including code 512 may be stored in the storage 508, or memory 506. In certain other embodiments, the program logic 510 may be implemented in the circuitry 502. Therefore, while FIG. 8 shows the program logic 510 separately from the other elements, the program logic 510 may be implemented in the memory 506 and/or the circuitry 502. The program logic 510 may include the program code discussed in this disclosure that facilitates the reorientation of one or more images in a display to stimulate a consistent and deliberately selected portion of the retina of a user of a computing device.

Using the processing resources of a resource 600 to execute software, computer-readable code or instructions, does not limit where this code can be stored. Referring to FIG. 8, in one example, a computer program product 600 includes, for instance, one or more non-transitory computer readable storage media 602 to store computer readable program code means or logic 604 thereon to provide and facilitate one or more aspects of the technique.

As will be appreciated by one skilled in the art, aspects of the technique may be embodied as a system, method or computer program product. Accordingly, aspects of the technique may take the form of an entirely hardware embodiment, an entirely software embodiment (including firmware, resident software, micro-code, etc.) or an embodiment combining software and hardware aspects that may all generally be referred to herein as a “circuit,” “module” or “system”. Furthermore, aspects of the technique may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable signal medium may include a propagated data signal with computer readable program code embodied therein, for example, in baseband or as part of a carrier wave. Such a propagated signal may take any of a variety of forms, including, but not limited to, electro-magnetic, optical or any suitable combination thereof. A computer readable signal medium may be any computer readable medium that is not a computer readable storage medium and that can communicate, propagate, or transport a program for use by or in connection with an instruction execution system, apparatus or device.

A computer readable storage medium may be, for example, but not limited to, an electronic, magnetic, optical, electromagnetic, infrared or semiconductor system, apparatus, or device, or any suitable combination of the foregoing. More specific examples (a non-exhaustive list) of the computer readable storage medium include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

Any combination of one or more computer readable medium(s) may be utilized. The computer readable medium may be a computer readable signal medium or a computer readable storage medium. A computer readable signal medium may include a propagated data signal with computer readable program code embodied therein, for example, in baseband or as part of a carrier wave. Such a propagated signal may take any of a variety of forms, including, but not limited to, electro-magnetic, optical or any suitable combination thereof. A computer readable signal medium may be any computer readable medium that is not a computer readable storage medium and that can communicate, propagate, or transport a program for use by or in connection with an instruction execution system, apparatus or device.

Program code embodied on a computer readable medium may be transmitted using an appropriate medium, including but not limited to wireless, wireline, optical fiber cable, RF, etc., or any suitable combination of the foregoing.

Computer program code for carrying out operations for aspects of the technique may be written in any combination of one or more programming languages, including an object oriented programming language, such as Java, Smalltalk, C++ or the like, and conventional procedural programming languages, such as the “C” programming language, PHP, ASP, assembler or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the technique are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions, also referred to as software and/or program code, may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the technique. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

In addition to the above, one or more aspects of the technique may be provided, offered, deployed, managed, serviced, etc. by a service provider who offers management of customer environments. For instance, the service provider can create, maintain, support, etc. computer code and/or a computer infrastructure that performs one or more aspects of the technique for one or more customers. In return, the service provider may receive payment from the customer under a subscription and/or fee agreement, as examples. Additionally or alternatively, the service provider may receive payment from the sale of advertising content to one or more third parties.

In one aspect of the technique, an application may be deployed for performing one or more aspects of the technique. As one example, the deploying of an application comprises providing computer infrastructure operable to perform one or more aspects of the technique.

As a further aspect of the technique, a computing infrastructure may be deployed comprising integrating computer readable code into a computing system, in which the code in combination with the computing system is capable of performing one or more aspects of the technique.

As yet a further aspect of the technique, a process for integrating computing infrastructure comprising integrating computer readable code into a computer system may be provided. The computer system comprises a computer readable medium, in which the computer medium comprises one or more aspects of the technique. The code in combination with the computer system is capable of performing one or more aspects of the technique.

Further, other types of computing environments can benefit from one or more aspects of the technique. As an example, an environment may include an emulator (e.g., software or other emulation mechanisms), in which a particular architecture (including, for instance, instruction execution, architected functions, such as address translation, and architected registers) or a subset thereof is emulated (e.g., on a native computer system having a processor and memory). In such an environment, one or more emulation functions of the emulator can implement one or more aspects of the technique, even though a computer executing the emulator may have a different architecture than the capabilities being emulated. As one example, in emulation mode, the specific instruction or operation being emulated is decoded, and an appropriate emulation function is built to implement the individual instruction or operation.

In an emulation environment, a host computer includes, for instance, a memory to store instructions and data; an instruction fetch unit to fetch instructions from memory and to optionally, provide local buffering for the fetched instruction; an instruction decode unit to receive the fetched instructions and to determine the type of instructions that have been fetched; and an instruction execution unit to execute the instructions. Execution may include loading data into a register from memory; storing data back to memory from a register; or performing some type of arithmetic or logical operation, as determined by the decode unit. In one example, each unit is implemented in software. For instance, the operations being performed by the units are implemented as one or more subroutines within emulator software.

Further, a data processing system suitable for storing and/or executing program code is usable that includes at least one processor coupled directly or indirectly to memory elements through a system bus. The memory elements include, for instance, local memory employed during actual execution of the program code, bulk storage, and cache memory which provide temporary storage of at least some program code in order to reduce the number of times code must be retrieved from bulk storage during execution.

Input/Output or I/O devices (including, but not limited to, keyboards, displays, pointing devices, DASD, tape, CDs, DVDs, thumb drives and other memory media, etc.) can be coupled to the system either directly or through intervening I/O controllers. Network adapters may also be coupled to the system to enable the data processing system to become coupled to other data processing systems or remote printers or storage devices through intervening private or public networks. Modems, cable modems, and Ethernet cards are just a few of the available types of network adapters.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or steps plus function elements in the descriptions below, if any, are intended to include any structure, material, or act for performing the function in combination with other elements as specifically noted. The description of the technique has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular uses contemplated.

1. A computer-implemented method comprising:
displaying, by one or more processors, at a first location in an interface of a computing device, one or more images comprising a fixation point;
obtaining, by the one or more processors, from an eye tracking device communicatively coupled to the one or more processors, a first indication that a user whose eye movement is being tracked by the eye tracking device has at least one eye focused on the fixation point;
based on the first indication, rendering, by the one or more processors, at a second location in the interface, a visual stimulus, wherein the second location is a fixed vertical distance and a fixed horizontal distance from the first location, and wherein based on being rendered at the second location, the stimulus stimulates a predefined location in a retina of the at least one eye of the user when the user has at least one eye focused on the fixation point;
obtaining, by the one or more processors, from the eye tracking device, a second indication that a user whose eye movement is being tracked by the eye tracking device does not have at least one eye focused on the fixation point;
identifying, by the one or more processors, based on utilizing the eye tracking device, a third location in the interface, wherein the third location comprises a focal point of the at least one eye of the user, in the interface; and
based on identifying the third location, reorienting, by the one or more processors, the one or more images such that the fixation point is at the third location and the stimulus is moved relative to the fixation point, maintaining the fixed vertical distance and the fixed horizontal distance.

2. The computer-implemented method of claim 1, wherein the interface comprises a head-mounted display.

3. The computer-implemented method of claim 1, wherein obtaining the first indication and obtaining the second indication is based on the one or more processors continuously monitoring the eye tracking device.

4. The computer-implemented method of claim 1, wherein obtaining the first indication and obtaining the second indication is based on the one or more processors intermittently obtaining data from the eye tracking device.

5. The computer-implemented method of claim 1, wherein obtaining the first indication comprises:
obtaining, by the one or more processors, video data captured by the eye tracking device;
analyzing, by the one or more processors, the video data to determine a focal point of the at least one eye in the interface;
comparing, by the one or more processors, the focal point to the fixation point; and
determining, by the one or more processors, that the focal point is equivalent to the fixation point.

6. The computer-implemented method of claim 1, wherein rendering the visual stimulus comprises displaying one or more images in a manner selected from the group consisting of: displaying an the one or more images sequentially in the interface at the second location, and pulsing the one or more image on and off at the second location.

7. The computer-implemented method of claim 6, wherein the stimulus is moved relative to the fixation point to a fourth location in the interface.

8. The computer-implemented method of claim 7, the method further comprising:
based on obtaining the second indication, stopping, by the one or more processors, the displaying of the one or more images.

9. The computer-implemented method of claim 8, further comprising:
based on reorienting the one or more images, resuming, by the one or more processors, the displaying of the one or more images at the fourth location.

10. The computer-implemented method of claim 1, wherein the reorienting is timed such that the user cannot perceive the reorienting.

11. A computer program product comprising:
a computer readable storage medium readable by one or more processors and storing instructions for execution by the one or more processors for performing a method comprising:
displaying, by the one or more processors, at a first location in an interface of a computing device, one or more images comprising a fixation point;
obtaining, by the one or more processors, from an eye tracking device communicatively coupled to the one or more processors, a first indication that a user whose eye movement is being tracked by the eye tracking device has at least one eye focused on the fixation point;
based on the first indication, rendering, by the one or more processors, at a second location in the interface, a visual stimulus, wherein the second location is a fixed vertical distance and a fixed horizontal distance from the first location, and wherein based on being rendered at the second location, the stimulus stimulates a predefined location in a retina of the at least one eye of the user when the user has at least one eye focused on the fixation point;
obtaining, by the one or more processors, from the eye tracking device, a second indication that a user whose eye movement is being tracked by the eye tracking device does not have the at least one eye focused on the fixation point;
identifying, by the one or more processors, based on utilizing the eye tracking device, a third location in the interface, wherein the third location comprises a focal point of the at least one eye of the user, in the interface; and
based on identifying the third location, reorienting, by the one or more processors, the one or more images such that the fixation point is at the third location and the stimulus is moved relative to the fixation point, maintaining the fixed vertical distance and the fixed horizontal distance.

12. The computer program product of claim 11, wherein the interface comprises a head-mounted display.

13. The computer program product of claim 11, wherein obtaining the first indication and obtaining the second indication is based on the one or more processors continuously monitoring the eye tracking device.

14. The computer program product of claim 11, wherein obtaining the first indication and obtaining the second indication is based on the one or more processors intermittently obtaining data from the eye tracking device.

15. The computer program product of claim 11, wherein obtaining the first indication comprises:
obtaining, by the one or more processors, video data captured by the eye tracking device;
analyzing, by the one or more processors, the video data to determine a focal point of the at least one eye in the interface;
comparing, by the one or more processors, the focal point to the fixation point; and
determining, by the one or more processors, that the focal point is equivalent to the fixation point.

16. The computer program product of claim 11, wherein rendering the visual stimulus comprises displaying one or more images in a manner selected from the group consisting of: displaying an the one or more images sequentially in the interface at the second location, and pulsing the one or more image on and off at the second location.

17. The computer program product of claim 16, wherein the stimulus is moved relative to the fixation point to a fourth location in the interface.

18. The computer program product of claim 17, the method further comprising:
based on obtaining the second indication, stopping, by the one or more processors, the displaying of the one or more images.

19. The computer program product of claim 18, the method further comprising:
based on reorienting the one or more images, resuming, by the one or more processors, the displaying of the one or more images at the fourth location.

20. A system comprising:
a memory;
one or more processors in communication with the memory;
an eye tracking device communicatively coupled to the one or more processors; and
program instructions executable by the one or more processors via the memory to perform a method, the method comprising:
displaying, by the one or more processors, at a first location in an interface of a computing device, one or more images comprising a fixation point;
obtaining, by the one or more processors, from the eye tracking device, a first indication that a user whose eye movement is being tracked by the eye tracking device has at least one eye focused on the fixation point;
based on the first indication, rendering, by the one or more processors, at a second location in the interface, a visual stimulus, wherein the second location is a fixed vertical distance and a fixed horizontal distance from the first location, and wherein based on being rendered at the second location, the stimulus stimulates a predefined location in a retina of the at least one eye of the user when the user has at least one eye focused on the fixation point;
obtaining, by the one or more processors, from the eye tracking device, a second indication that a user whose eye movement is being tracked by the eye tracking device does not have the at least one eye focused on the fixation point;
identifying, by the one or more processors, based on utilizing the eye tracking device, a third location in the interface, wherein the third location comprises a focal point of the at least one eye of the user, in the interface; and
based on identifying the third location, reorienting, by the one or more processors, the one or more images such that the fixation point is at the third location and the stimulus is moved relative to the fixation point, maintaining the fixed vertical distance and the fixed horizontal distance.
